(19) United States
(12) Patent Application Publication
(10) Pub. No.: US 2026/0141993 A1
(43) Pub. Date: May 21, 2026

(54) Systems and Methods for Healthcare Visit Follow-Up and Education

(71) Applicant: Victor Joseph Mazza, Hasbrouck Heights, NJ (US)

(72) Inventor: Victor Joseph Mazza, Hasbrouck Heights, NJ (US)

(21) Appl. No.: 19/391,496

(22) Filed: Nov. 17, 2025

Related U.S. Application Data
(60) Provisional application No. 63/721,675, filed on Nov. 18, 2024.

(51) Int. Cl. G16H 10/20; G16H 40/67

(52) U.S. Cl. CPC G16H 10/20; G16H 40/67

(57) ABSTRACT

A computing system includes a medical application for patient follow-up and education after a healthcare visit. The computing system includes a database with educational materials relating to diagnosis, medications, procedures, etc. A computing system obtains a selection from a healthcare provider, using a provider application or web portal, of the educational materials relating to a patient diagnosis or treatment plan. The computing system provides access to the selected educational materials to the patient using a patient application or web portal. The computing system generates a comprehension test relating to the educational materials and a symptom questionnaire relating to the patient diagnosis or treatment plan. The computing device may also generate a compliance questionnaire relating to patient compliance with the treatment plan. The computing device receives a patient response to the comprehension test, the symptom questionnaire and the compliance questionnaire and provides the patient response to the healthcare provider.

### CROSS REFERENCE TO RELATED APPLICATIONS

This application is a U.S. non-provisional patent application claiming priority under 35 U.S.C. § 119(e) to U.S. provisional patent application No. 63/721,675, filed Nov. 18, 2024, entitled, “SYSTEMS AND METHODS FOR HEALTHCARE VISIT FOLLOW-UP AND EDUCATION,” which is hereby expressly incorporated by reference as if fully set forth herein.

### COPYRIGHT NOTICE

A portion of the disclosure of this patent document contains material which is or may be subject to copyright protection. The copyright owner has no objection to the facsimile reproduction by anyone of the patent document or the patent disclosure, as it appears in the Patent and Trademark Office patent file or records, but otherwise reserves all copyrights whatsoever.

### FIELD

Embodiments of the present application generally relate to a computer network and application that provides customized follow-up and education for patients following a healthcare visit.

### BACKGROUND OF THE INVENTION

Healthcare-patient visits can be quite complex and time consuming. Healthcare visits take place in different settings—locations include but are not limited to telehealth, outpatient medical office, outpatient urgent care, emergency department, inpatient hospital and scheduled surgical procedures. In the current environment, patients are scheduled based on a limited allotted time, e.g., based on the visit type: new patient versus established patient. This allotted time includes reviewing the relevant patient information and available electronic health record, talking to the patient, reviewing medications, examining the patient and generating a treatment plan. In addition, one of the most important parts of the visit is patient education. This includes educating the patient about their diagnosis, describing options for management, and explaining the treatment plan. Some patients have quite complex conditions and may have an extensive medical history, a very long chart to review, numerous medications and prior testing/results. The diagnosis and treatment plan may be quite complex and require a significant amount of time. This complexity may leave little to no time for patient education during the limited allotted time for the patient's visit.

Another important part of a healthcare visit includes a patient asking questions of the healthcare professional, listening to answers and instructions and comprehending the diagnosis and treatment plan. However, a patient may be hampered by time constraints (patient feeling rushed), patient nervousness and frozen thought process. In addition, advanced age or language barriers may also prevent a patient completely understanding the process. The patient often leaves their medical visit and later has no recollection of the instructions and education provided during the visit or has new questions. The patient may feel unfulfilled because the healthcare visit didn't provide all the information the patient wanted to discuss. This leads to uncertainty, questions for caregivers, anxiety and lack of consistency of care. The patient may feel the medical visit was an ineffective encounter and inefficient use of time.

Furthermore, the healthcare providers (physicians, nurse practitioners, physician's assistants, nurses, etc.), are not able to reassess the patient until the next appointment. Though a patient may call with questions or submit questions through current medical applications, such as MyChart® application, the healthcare providers have no way to determine whether the patient has a true understanding of their medical condition and treatment plan or whether the patient is following the treatment plan or whether the treatment plan is improving the medical condition. Current applications, such as the MyChart application, only provide the medical test results and prescription information without further explanation. For at least these reasons, a gap exists in patient care with the current encounter system and medical applications.

As such, there is a need for an improved medical application that provides patient education and patient follow up after healthcare visits.

### BRIEF SUMMARY OF THE INVENTION

In one aspect, a computing system includes a medical application and comprises at least one network interface configured for communication over one or more networks; at least one memory device, wherein the at least one memory device includes an education material database that stores a plurality of educational materials; and at least one processing device, wherein the processing device is operatively coupled to the at least one memory device and wherein the at least one memory device stores instructions that, when executed by the at least one processing device, configures the computing system to obtain, from a healthcare provider device associated with a healthcare provider, a selection of one or more of the plurality of educational materials for a patient, wherein the one or more selected educational materials relate to a patient diagnosis or treatment plan; provide access to the one or more selected educational materials to a patient device associated with the patient; generate a comprehension test relating to the patient diagnosis or treatment plan and obtain a response to the comprehension test from the patient device; generate a symptom questionnaire relating to the patient diagnosis or treatment plan and obtain a response to the symptom questionnaire from the patient device; generate a compliance questionnaire relating to patient compliance with the treatment plan and obtain a response to the compliance questionnaire from the patient device; and provide access to the response to the comprehension test, the response to the symptom questionnaire and the response to the compliance questionnaire to the healthcare provider device.

In another aspect, a computing system including a medical application comprises at least one network interface configured for communication over one or more networks; at least one memory device, wherein the at least one memory device includes an education material database that stores a plurality of educational materials; and at least one processing device, wherein the processing device is operatively coupled to the at least one memory device and wherein the at least one memory device stores instructions that, when executed by the at least one processing device, configures the computing system to: obtain, from a healthcare provider device associated with a healthcare provider, a confirmation of a scheduled procedure for a patient and a procedure date; obtain, from the healthcare provider device, a selection of one or more of the plurality of educational materials for the patient, wherein the one or more selected educational materials include risk and benefits of the scheduled procedure and pre-procedure instructions and post-procedure instructions; provide access to the one or more selected educational materials to a patient device associated with the patient; generate a comprehension test relating to the one or more selected educational materials and obtain a response to the comprehension test from the patient device; and generate an informed consent questionnaire and obtain a response from the patient device, wherein the informed consent questionnaire relates to the scheduled procedure.

In yet another aspect, a method of a computing system comprises obtaining, from a healthcare provider device associated with a healthcare provider, a selection of one or more of a plurality of educational materials for a patient, wherein the one or more selected educational materials relate to a patient diagnosis or treatment plan; providing, by the computing system, access to the one or more selected educational materials to a patient device associated with the patient, wherein the one or more selected materials are stored in a database on a memory device of the computing system; generating, by the computing system, a comprehension test relating to the patient diagnosis or treatment plan for display on the patient device and obtaining a response to the comprehension test from the patient device; generating, by the computing system, a symptom questionnaire relating to the patient diagnosis or treatment plan for display on the patient device and obtaining a response to the symptom questionnaire from the patient device; generating, by the computing system, a compliance questionnaire relating to patient compliance with the treatment plan for display on the patient device and obtaining a response to the compliance questionnaire from the patient device; and providing, by the computing system, access to the response to the comprehension test, the response to the symptom questionnaire and the response to the compliance questionnaire to the healthcare provider device.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. To illustrate embodiments, there is shown in the drawings various examples and/or preferred embodiments. It should be understood, however, that the invention as claimed is not limited to the embodiments, examples, arrangements and instrumentalities shown in the drawings.

The subject application references certain processes and methods which are presented as a series of ordered steps. The steps described with respect to these processes are not to be understood as enumerated consecutive lists but could be performed in various orders while still embodying the invention described herein. Additional and/or alternate steps may be included in such processes as well.

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “an appliance” may include a plurality of appliances. Thus, for example, a reference to “a method” includes one or more methods, and/or steps of the type described herein and/or which will become apparent to those persons skilled in the art upon reading this disclosure.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, constructs, and materials are now described. Where there are discrepancies in terms and definitions used in references that are incorporated by reference, the terms used in this application shall have the definitions given herein.

FIG. 1 depicts a schematic block diagram of an exemplary network environment including a medical application (MA) computing system, in accordance with one or more embodiments herein.

FIG. 2 depicts a schematic block diagram of an exemplary MA computing device in accordance with one or more embodiments herein.

FIG. 3 depicts a flow chart of an exemplary computing method of the MP computing system in accordance with one or more embodiments herein.

FIGS. 4A-C depicts exemplary symptom questionnaires for a patient generated by the MA computing system in accordance with one or more embodiments herein.

FIG. 5A depicts a flow chart of a method of a provider application or web portal of the MPA computing system in accordance with one or more embodiments herein.

FIG. 5B depicts a flow chart of a method of the patient application or web portal of the MPA computing system in accordance with one or more embodiments herein.

FIG. 5C depicts a flow chart of another method of the provider application or web portal of the MPA computing system in accordance with one or more embodiments herein.

FIG. 6 depicts a flow chart of a method of the MA computing system for analysing data related to comprehension tests in accordance with one or more embodiments herein.

FIG. 7 depicts a flow chart of a method of the MA computing system for analysing data related to treatment compliance questionnaires in accordance with one or more embodiments herein.

FIG. 8 depicts a flow chart of a method of the MA computing system for analysing data related to the patient surveys in accordance with one or more embodiments herein.

FIG. 9A depicts a flow chart of a method of the MPA computing system when the treatment plan includes a patient procedure in accordance with one or more embodiments herein.

FIG. 9B depicts a flow chart of a method of the MPA computing system prior to a patient procedure in accordance with one or more embodiments herein.

FIG. 9C depicts a flow chart of a method of the MPA computing system after a patient procedure in accordance with one or more embodiments herein.

FIG. 9D depicts a flow chart of a method of the provider application or web portal of the MPA computing system in accordance with one or more embodiments herein.

FIG. 10 depicts a flow chart of a method of the MPA computing system for obtaining informed consent of a patient in accordance with one or more embodiments herein.

FIG. 11 illustrates a schematic block diagram of an embodiment of a user device in accordance with one or more embodiments herein.

FIG. 12 illustrates a schematic block diagram of another embodiment of a user device in accordance with one or more embodiments herein.

### DETAILED DESCRIPTION OF THE INVENTION

The subject application references certain processes which are presented as series of ordered steps. The steps described with respect to these processes are not to be understood as enumerated consecutive lists but could be performed in various orders while still embodying the invention described herein.

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “an appliance” may include a plurality of appliances. Thus, for example, a reference to “a method” includes one or more methods, and/or steps of the type described herein and/or which will become apparent to those persons skilled in the art upon reading this disclosure.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, constructs, and materials are now described. All publications mentioned herein are incorporated herein by reference in their entirety. Where there are discrepancies in terms and definitions used in references that are incorporated by reference, the terms used in this application shall have the definitions given herein.

### Overview

The specification describes various embodiments of computer systems and applications that support follow-up and patient education after a healthcare visit. The system extends a healthcare visit by providing educational materials to patients about their diagnosis and treatment plan to ensure the patient's understanding of and compliance with the treatment plan and providing an interface for a patient to submit questions to the healthcare provider. The system obtains confirmation from the patient that the educational materials have been received and understood. The system also provides a platform to obtain patient feedback on the effectiveness of the treatment plan and their ongoing symptoms and condition after the healthcare visit. The medical provider receives the information and decides whether intervention is needed, e.g., such as a change to the treatment plan. The system further obtains confirmation that a patient is following their current or modified treatment plan and responses to a patient satisfaction survey. The medical application focuses on all parts of the patient encounter timeline, delivers information about the visit and future intervention, and allows for bidirectional information between the patient and healthcare provider between visits.

### Detailed Embodiments

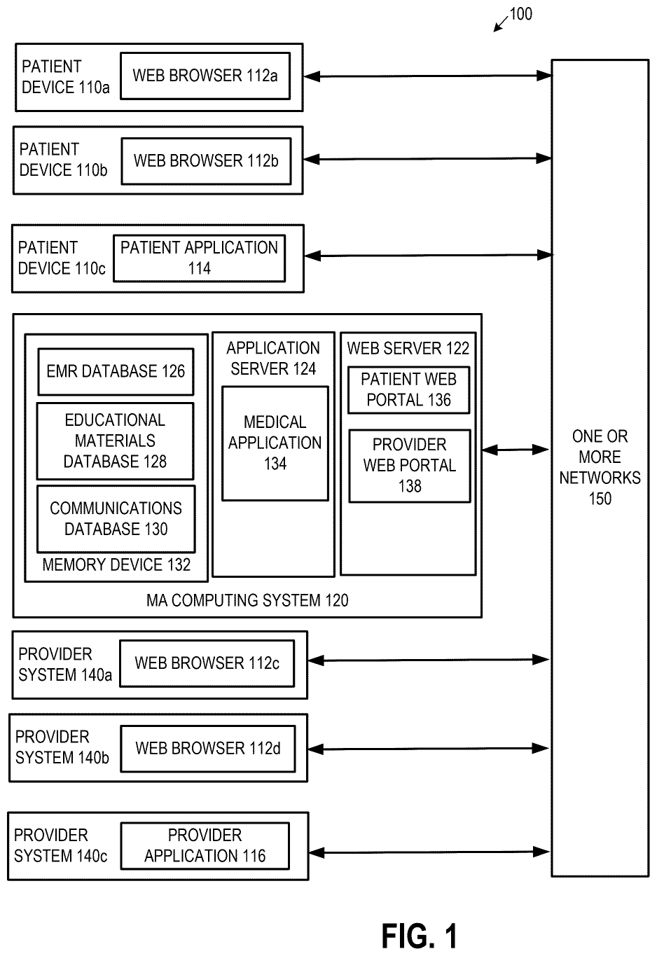

FIG. 1 depicts a schematic block diagram of an exemplary network environment 100 for implementing a computing system 120 that supports a medical application (MA) 134 in accordance with one or more embodiments herein. The network environment 100 includes a plurality of patient devices 110a-c that are configured to communicate with the MA computing system 120. The patient devices 110a-c may include computing devices, such as personal computers (“PCs”), server computers, handheld or laptop devices, cell phones, smartphones, tablets, minicomputers, or other devices including at least one processing device and at least one memory device. The patient devices 110a-c may also have input device(s) and output devices such as keyboard, mouse, pen, mouse, voice input device, touch input device, a display, speakers, LED light, etc. In an embodiment, one or more of the patient devices 110a-b are web-enabled devices configured with a web browser 112a-b to communicate with a web server 122 of the MA computing system 120. Additionally, and/or alternatively, one or more of the patient devices 110c include a patient application 114 that communicates using an application program interface (API) with the application server 124 of the MA computing system 120.

One or more provider systems 140a-c include one or more computing devices, such as server computers, database servers, personal computers (“PCs”), handheld or laptop devices, cell phones, smartphones, tablets, minicomputers, or other devices including at least one processing device and at least one memory device. One or more of the provider systems 140a-b may include web browsers 112c-d to communicate with the web server 122 of the MA computing system 120. Alternatively, or additionally, the provider systems 140c include a provider application 116 that communicates using an application program interface (API) with the application server 124 of the MA computing system 120.

In an embodiment, the web server 122 hosts one or more web portals, such as a patient web portal 136 and a provider web portal 138. The web portals are web-based platforms that provide graphical interfaces for and access to the functionality and services of the medical application 134 and MA computing system 120 as well as access to data in all or portions of one or more of the databases of the MA computing system 120. In an embodiment, the web portals 136, 138 are configured to interact with the one or more web browsers 112a-d on the patient devices 110a-c and provider systems 140a-c via a HyperText Transfer Protocol (“HTTP”) and/or a secure version (e.g., “https”) of a related Uniform Resource Locator (“URL”). An application server 124 may be accessible by the web server 122 to help respond to the web browsers 122a-d, using the publicly addressable URL. The application server 124 may thus provide a software as a service (SaaS) type offering for the medical application. The web server 122 may include a network interface card (NIC) that includes one or more transceivers for wireless and/or wired network communications over the one or more networks 150. The NIC may also include authentication capability that requires an authentication process prior to allowing access to one or more of the web portals. For example, the authentication may include a login with a username and password, biometric identification, or other processes. The NIC may also include firewall, gateway, and proxy server functions.

In another embodiment, APIs on the patient application 114 and the provider application 116 communicate directly with the application server 124 in a client/server type architecture. The application server 124 may include a NIC to communicate with the one or more networks 150 directly. Alternate methods of computing device/server communications may be substituted without departing from the scope hereof. Though a single web server 122 and application server 124 are illustrated, multiple web and/or application servers may be implemented to perform the functions described herein.

The MA computing system 120 may further include at least one memory device 132 that stores one or more databases, such as an electronic medical records (EMR) database 126, educational materials database 128, and communications database 130, as explained in more detail herein. The memory device 132 may include a server or other device including RAM, ROM, electrically erasable programmable read-only memory (“EEPROM”), flash memory or other memory technology, CD-ROM, digital versatile disks (“DVD”) or other optical storage, magnetic cassettes, magnetic tape, magnetic disk storage or other magnetic storage devices, cloud devices, or any other medium which can be used to store the desired information and which can accessed by the application server 124.

The one or more networks 150 may include, e.g., a wide area network (WAN) and/or a wireless wide area network (Wireless WAN). For example, the WAN may include the Internet, service provider network, satellite WAN, or a combination of one or more thereof. The Wireless WAN may include a cellular network, such as a 4G or 5G cellular network. The WAN or Wireless WAN are communicatively coupled directly to the patient devices 110a-c or coupled to the devices through an edge network and/or local area network (LAN), e.g., including a router, bridge, cable network, and/or a WLAN access point (AP) or another type of network or devices. The one or more networks 150 work to communicatively couple the patient devices 110a-c and the provider systems 140a-c to the MA computing system 120. Alternate networks and/or methods of communicating information may be substituted without departing from the scope hereof.

The exemplary network environment 100 provides a technical solution to the technical problem of effective patient education and follow-up. The network environment 100 depicts special purpose computing systems and devices that perform various functions and methods described herein. The depicted network environment 100 is only one example of a suitable computing environment and is not intended to suggest any limitation as to the scope of use or functionality. Numerous other special purpose computing system environments or other configurations may be used. Though shown separately, the databases may be included in one or more of their respective servers. The devices, servers and databases shown in FIG. 1 are merely exemplary and one or more of the devices, servers and/or databases may be omitted or added or combined without departing from the scope of the present invention.

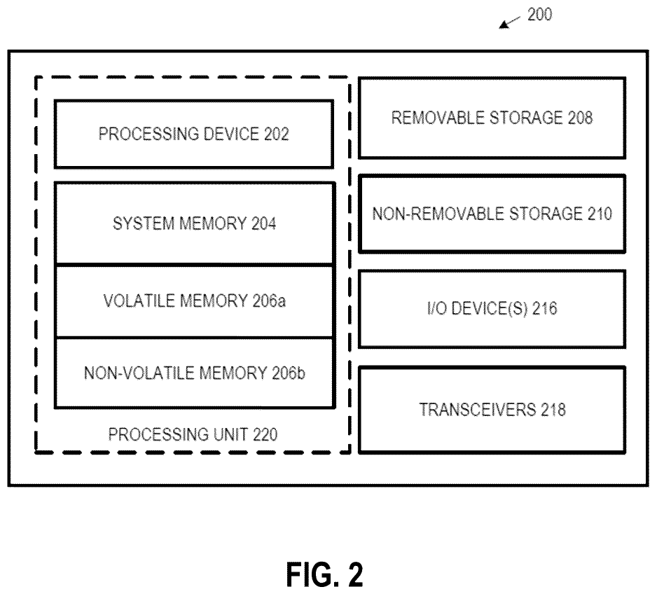

FIG. 2 depicts a schematic block diagram of an exemplary computing device 200 in accordance with one or more embodiments herein. The computing device 200 shows a basic configuration of the patient devices 110a-c, web server 112, application server 124, and provider systems 140a-c. The computing device 200 includes a processing unit 220 having at least one processing device 202 and at least one system memory 204. Depending on the exact configuration and type of the computing device, system memory 204 may include volatile memory 206a (such as, random access memory (“RAM”)) 206a, non-volatile memory 206b (such as read-only memory (“ROM”), flash memory, etc.), or some combination of the two. Computing device 200 may may include additional storage, such as removable storage 208 or non-removable storage 210, including, but not limited to, magnetic or optical disks or tape, thumb drives, and external hard drives, RAM, ROM, electrically erasable programmable read-only memory (“EEPROM”), flash memory or other memory technology, CD-ROM, digital versatile disks (“DVD”) or other optical storage, magnetic cassettes, magnetic tape, magnetic disk storage or other magnetic storage devices, or any other medium or any other available storage media that can be accessed by computing device 200. The system memory 204 and/or the removable storage 208 and/or non-removable storage 210 includes computer-readable instructions, data structures, program modules, application-program interfaces (“APIs”), etc. that when executed by the processing device 202, causes the computing device 200 to perform one or more functions described herein. Generally, program modules include routines, programs, objects, components, data structures, etc. that perform tasks or implement particular abstract data types. Such programs may be implemented in a high-level procedural or object-oriented programming language to communicate with the computer device 200. However, the program(s) can be implemented in assembly or machine language, if desired. In any case, the language may be a compiled or interpreted language and combined with hardware implementations.

The computing device 200 further includes at least one input/output (I/O) device 216, such as a display or touch screen, touch pad, keyboard, printer, speaker, mouse, etc. The computing device 200 may also include one or more transceivers 218, such as a wireless or wired transceiver, that allows the computing device 200 to communicate with other devices over one or more networks. Such one or more transceivers include computer-readable instructions, data structures, program modules and/or other data, to transmit a modulated data signal such as a carrier wave or other transport mechanism and includes any information delivery media. By way of example, and not limitation, communication media includes wired media such as a wired network or direct-wired connection, and wireless media such as acoustic, radio frequency (“RF”), infrared and other wireless media. In addition to that described herein, computing device 200 can be any web-enabled handheld device (e.g., cell phone, smart phone, or the like) or personal computer including those operating via Android™, Apple®, and/or Windows® mobile or non-mobile operating systems.

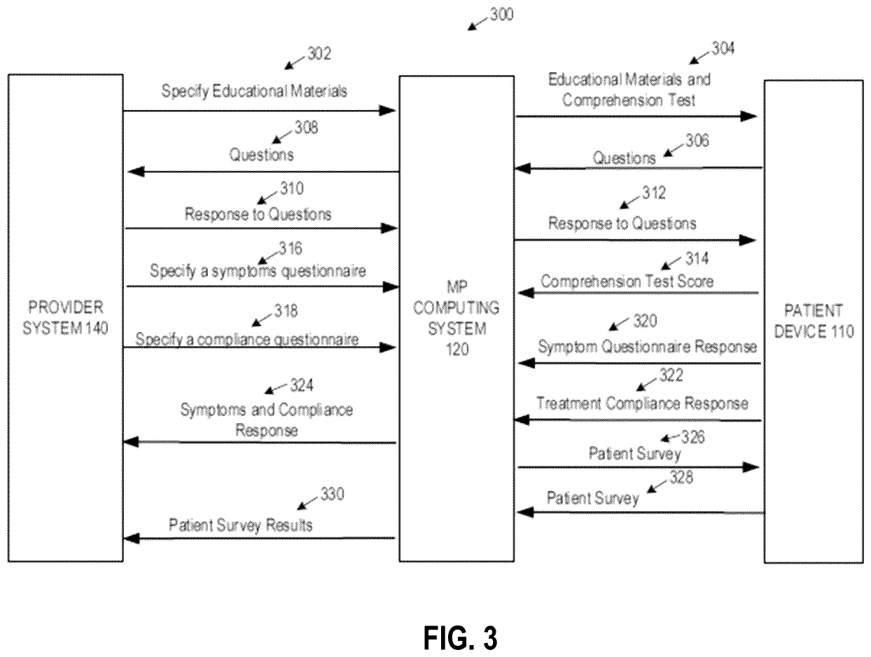

FIG. 3 depicts a flow chart of an exemplary method 300 of the MP computing system 120 for patient education and follow-up. During a healthcare visit, a patient is diagnosed with one or more conditions and a treatment plan is recommended by the healthcare provider. After the healthcare visit, at 302, the healthcare provider accesses the MA computing system 120, e.g., using a web browser 112 or provider application 116, and views available educational materials stored in the educational materials database 128. The healthcare provider selects one or more of the educational materials relevant to the diagnosis and/or treatment plan of the patient. The healthcare provider may also record a video with a personal message to the patient. The MA computing system 120 provides the selected educational materials and any video to the patient device 110, e.g., using a web browser 112 or patient application 114.

The educational materials may include data files, audio files, and/or video files. The educational material may be created by a third party and stored on the educational materials database 128. Alternatively, or additionally, the educational materials may be created by the healthcare provider and uploaded to the MA computing system 120 by the provider system 140. The educational materials may include information on the diagnosis, the treatment plan, upcoming procedures, etc. The specific content is selected by the healthcare provider and may include a slide presentation, video recording, audio file, data sheets, articles or any specific medical grade handouts that the healthcare provider wants the patient to have. In addition, the education materials may include or be followed by a comprehension test. The comprehension test may include a small number of questions to determine whether the patient understands their diagnosis and treatment plan. The comprehension test may include questions about the diagnosis, treatment plan, medications, procedures, testing, treatment options, etc. The comprehension test may be presented as a slide show with multiple choice questions.

At 306, the patient device 110 uploads a question to the MP computing system 120. The provider system 140 receives the question from the MP computing system 120 at 308 and uploads a response to the question to the MP computing system 120 at 310. The MP computing system provides the response to the patient device 110 at 312, e.g., through the web browser 112 or patient application 114. The response may be written, a voice recording or a video of the healthcare provider answering the question. Though steps 306-312 are shown after the education materials are received, the patient may transmit a question through the MP computing system 120 at any time. The ability to submit questions to and receive responses from the healthcare provider through the MP computing system 120 helps boost a patient's confidence in and compliance with the treatment plan.

At 314, the patient completes the comprehension test, and the test score is stored on the MP computing system 120, e.g., in the communications database 130. The provider system 140 may access the comprehension test score and determine whether the test score is at least a minimum value. If not, the provider system 140 may specify additional education materials for the patient, call the patient to discuss further, or request another healthcare visit for the patient. When a patient has a new diagnosis of a complex condition, such as diabetes, it is especially vital that the patient understands the condition and its treatment. The healthcare provider thus may require a higher minimum score on the comprehension test for certain serious conditions than a less serious condition.

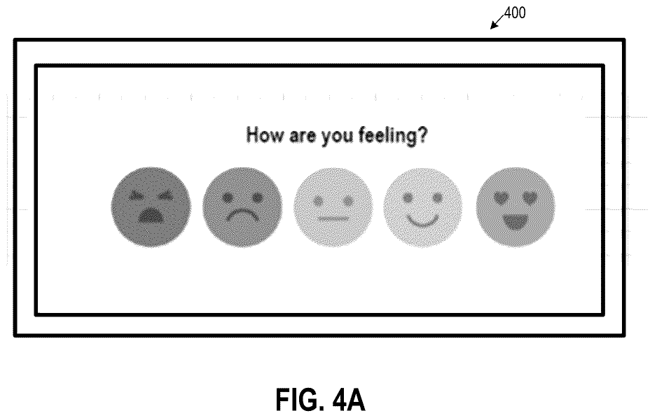

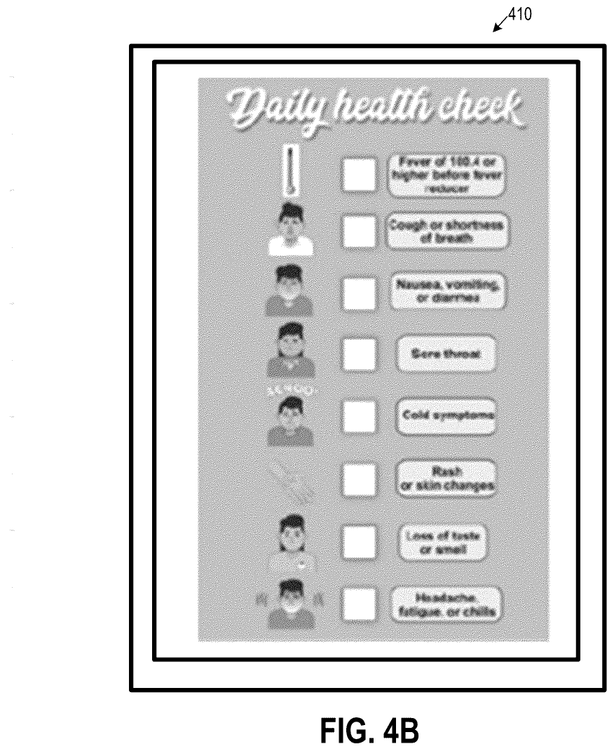

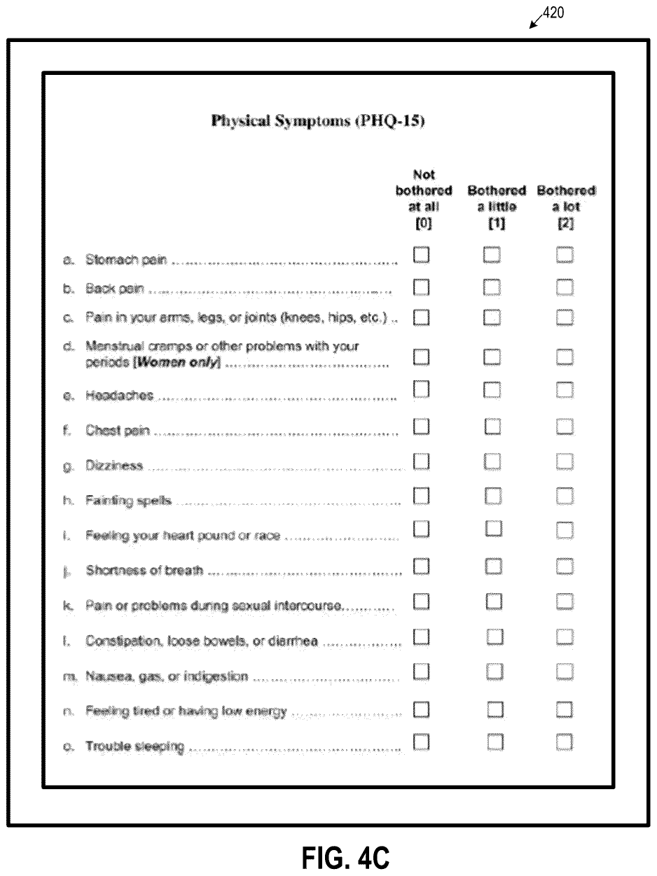

At 316, the provider system 140 indicates a symptom questionnaire and time period(s) for completion by the patient to the MP computing system 120. The symptom questionnaire may be created by a third party and stored in the educational materials database 128. Alternatively, or additionally, the symptom questionnaire may be created by the healthcare provider and uploaded to the MA computing system 120. The symptom questionnaire may vary depending on the diagnosis and treatment plan. For example, the symptom questionnaire may request information on the occurrence of any possible side effects of a prescribed medication, current pain level of the patient, blood pressure, heart rate, or temperature of the patient, etc. The healthcare provider may also indicate the time period(s) for completion of the symptom questionnaire, e.g., such as daily for one week after the healthcare visit, or just once after three days from the healthcare visit. FIGS. 4A-C illustrate exemplary symptom questionnaires, as described in more detail below. The questionnaires are designed to determine whether the treatment plan is effective in treating the medical condition(s) of the patient and/or whether the medications are having any intolerable side effects.

At 318, the provider system 140 indicates a treatment compliance questionnaire and time period(s) for completion by the patient. The treatment compliance questionnaire may be created by a third party and stored in the educational materials database 128. Alternatively, or additionally, the treatment compliance questionnaire may be created by the healthcare provider and uploaded to the MA computing system 120. Again, the treatment compliance questionnaire depends on the treatment plan. The questionnaire may need to be completed daily for a week after the appointment or completed just once after five days from the visit. The treatment questionnaire is designed to assess whether the patient is following the treatment plan, e.g., has the patient obtained their medication, is the patient taking the medication as prescribed, or whether the patient has scheduled any requested testing or procedures.

At 324, the provider system 140 accesses the patient's responses to the symptom questionnaire and the treatment compliance questionnaire. The healthcare provider may decide next steps depending on the responses, such as changing the treatment plan, calling the patient, requesting another visit, etc.

At 326, the MP computing system 120 generates a patient survey that requests feedback on satisfaction with the medical care. For example, the patient survey may ask the overall understanding of the diagnosis and treatment plan, the awareness of treatment options and overall satisfaction. The MP computing system 120 receives the completed patient survey at 328 and processes the results. The patient surveys may be anonymous so that the provider system 140 only has access to average patient survey results at 330.

FIGS. 4A-C illustrate schematic block diagrams of graphical user interfaces (GUIs) of symptom questionnaires generated using the MA computing system 120, and in specific the patient web portal 136, in accordance with one or more embodiments herein. The GUIs in FIGS. 4A-C are merely exemplary to illustrate types of data fields displayed and functionality of the system. The fields, icons and data described with respect to these GUIs may be included on additional and/or alternate GUIs or in other ways than described herein.

Referring to FIG. 4A, it illustrates a GUI 400 of a simplest questionnaire generated by one or more patent devices 110a-c using data from the MA computing system 120. This GUI 400 requests a simple indication of how the patient is feeling. The questionnaire 400 includes a subjective range, such as the different emoji faces, or may request a patient to grade how well they are feeling on a scale of 1 to 5, etc. FIG. 4B illustrates a GUI 410 including a more detailed questionnaire 410 that requests more objective information about symptoms, such as fever, cough or shortness of breath, nausea, vomiting or diarrhea, sore throat, cold symptoms, rash or skin changes, loss of taste or smell, headache, fatigue or chills. This second questionnaire 410 may be requested after a patient presents with COVID, flu, or a cold to ensure that the patient is improving, and that the treatment plan is working. FIG. 4C illustrates a GUI 420 including an even more detailed questionnaire requesting information on even more possible symptoms. In another embodiment, one or more of the symptom questionnaires 400, 410 and 420 may also be presented to a patient prior to their healthcare visit by the MPA computing system 120. The MPA computing system 120 may store the completed symptom questionnaire from prior to the healthcare visit for comparison to a symptom questionnaire completed by the patient after the treatment plan has begun. This comparison may provide a measure of the progress of the patient and the effectiveness of the treatment plan.

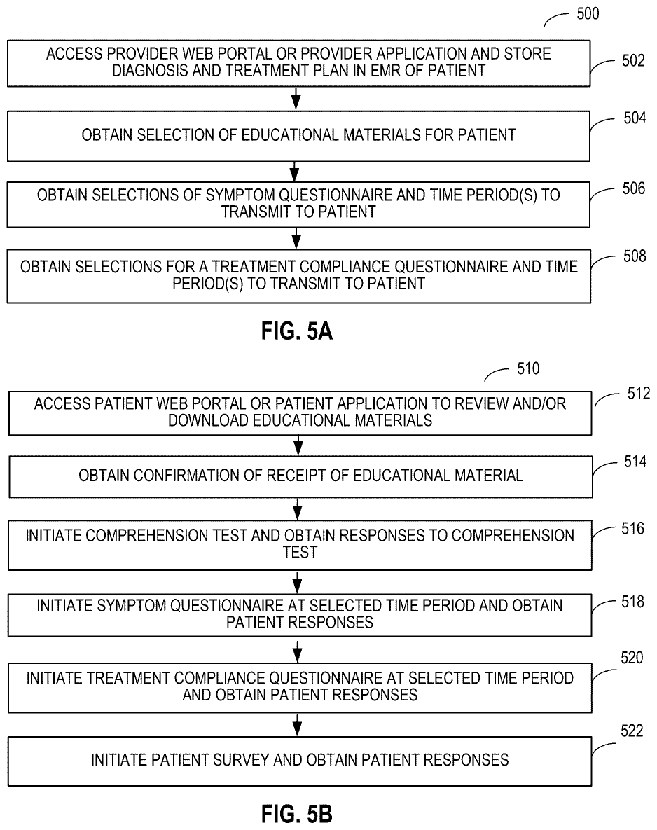

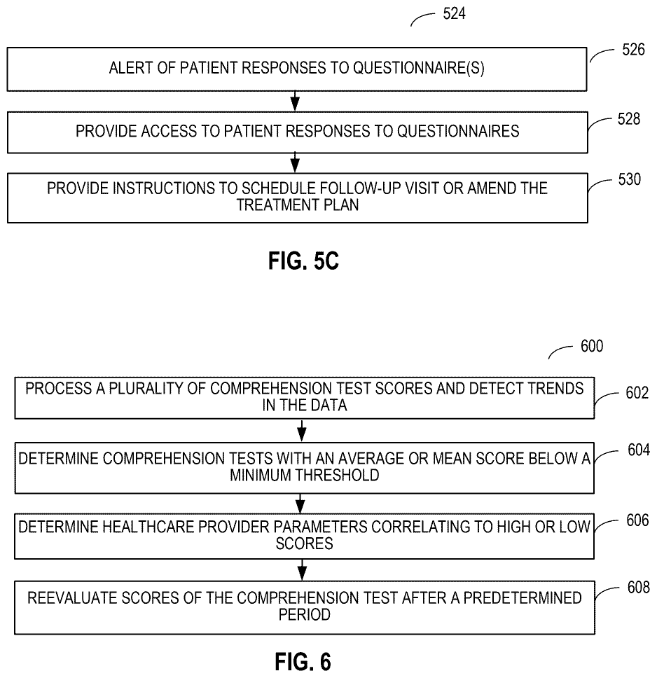

FIG. 5A illustrates a flow chart of a method 500 of the MPA computing system 120. In this example, a healthcare provider examines a patient at an office visit and diagnoses an otitis media or a middle ear infection in the patient and advises a treatment plan of an antibiotic. At 502, the diagnosis and prescription are recorded in the electronic medical record (EMR) of the patient, such as the EMR database 126. At 504, the healthcare provider accesses the MA computing system 120, e.g., using the web browser 112c-d or provider application 116 on a provider system 140a-c, and selects education materials for the patient. The educational materials database 120 may be searchable by keyword (such as the diagnosis or medication) and/or organized in folders by diagnosis and treatment, such as in a folder labeled “otitis media” or “antibiotic”. The educational materials may relate to one or more of: the diagnosis, treatment plan, medications, indication for medications, what to expect, or how to deal with the natural course of the illness and/or medication side effects. The healthcare provider may also prepare written materials and upload them to the MA computing system 120 for the patient. In addition, the healthcare provider may also record a video with a personal message for the patient. The MA computing system 120 includes functionality to record and store audio and/or video files by the healthcare provider and patient.

At 506, the healthcare provider selects a symptom questionnaire and time period(s) to transmit the questionnaire to the patient, and at 508, the healthcare provider selects the treatment compliance questionnaire and time period(s) to transmit to the patient. The MA computing system 120 obtains the selections and provides access to the educational materials and questionnaires to the patient using the patient web portal 136 and/or patient application 114.

FIG. 5B illustrates a flow chart of another method 510 of the MPA computing system 120. Continuing the example from FIG. 5A, at 512, the patient accesses the educational materials relating to the ear infection diagnosis, e.g., using the patient application 114 or patient web portal 136 of the MA computing system 120. The patient acknowledges receipt of the educational materials, and the MA computing system records the confirmation of receipt at 514. Upon selection or request of the patient, or at a predetermined time period (such as 1-2 days) after such confirmation, the MA computing system 120 initiates a comprehension test over the provided educational materials and obtains the responses to the questions at 516. At 518, the MA computing system 120 initiates a symptom questionnaire at the selected time period and obtains the patient responses. At 520, the MA computing system 120 initiates the treatment compliance questionnaire at the selected time period and obtains the patient responses. At 520, the MA computing system 120 initiates the patient survey and obtains the patient responses. In this example, the patient does not initiate any questions to the healthcare provider. The MPA computing system 120 may provide alerts to a patient, e.g., when education materials are available through the patient web portal 136, when a symptom questionnaire needs to be completed, when a treatment compliance questionnaire needs to be completed, etc. The alerts may be emails, text messages, notifications through the application, etc.

The MA computing system 120 processes the patient responses to the comprehension tests, questionnaires, and patient survey and generates one or more reports, as discussed in more detail herein. The MA computing system 120 also provides access to the patient responses and/or scores to the healthcare provider.

FIG. 5C illustrates a flow chart of another method 524 of the provider application 116 or web portal 138 of the MPA computing system 120. The MPA computing system 120 may provide an alert that new patient responses are available at 526. The healthcare provider then accesses the provider web portal 138 or provider application 116 and reviews the responses and/or scores of the patient questionnaires at 528. In this example, the comprehension score is 100% and the treatment compliance questionnaire indicates that the patient has followed the treatment plan, e.g., the prescription has been filled and the patient is taking the medication as prescribed. However, the symptom questionnaire indicates that the patient still has symptoms and/or is still not feeling well. The MPA computing system 120 may flag the patient response to the symptom questionnaire or generate an alert to the healthcare provider. The healthcare provider is thus promptly notified of the patient's condition. The healthcare provider thus has the opportunity to communicate with the patient to schedule another visit or amend the treatment plan at 530. For example, the healthcare provider may indicate that a new prescription for a different antibiotic has been submitted to the patient's pharmacy and for the patient to stop the first antibiotic and begin the new antibiotic.

The MPA computing system 120 thus provides an alert of ongoing symptoms which allows the healthcare provider to intervene promptly. The prompt intervention helps the patient feel better more quickly, prevents worsening of the infection/illness, and prevents or decreases emergency room visits.

In another example, a patient is newly diagnosed with atrial fibrillation at a healthcare visit. The diagnosis may seem overwhelming to the patient and difficult for them to understand the terminology, implications of the diagnosis, indications for the medications, etc. After the visit, the healthcare professional may access the MA computing system 120 using the provider web portal 138 or provider application 116 and record a personal video to the patient about the diagnosis and reasons for the medications. The healthcare provider may select educational materials, such as videos or written materials, about the diagnosis, the plan of care (rate vs. rhythm control) and need for medications. For example, the patient may not understand why anticoagulation medication is needed for a diagnosis of atrial fibrillation and may not want to spend the money on the medication or risk the side effects. The materials may provide a more thorough explanation of the increased risk of blood clots and stroke with atrial fibrillation. Understanding this information may be essential for a patient's compliance with the treatment plan and prevention of adverse outcomes. The patient can spend time at home, at their own rate, reviewing the materials. The personal video by the healthcare provider may also comfort the patient and give the patient more confidence in the treatment plan. In addition, the educational materials are approved by the healthcare provider and are not misleading such as materials found on the internet or social media. The patient may submit any questions and complete the questionnaires and surveys in the days ahead. The healthcare provider accesses the MA computing system 120 and reviews the scores, responses and answers any submitted questions.

FIG. 6 illustrates a flow chart of a method 600 of the MA computing system 120 for analysing data relating to the comprehension tests. At 602, the MA computing system 120 collates data from a plurality of comprehension tests completed by patients across the country and determines various trends in the data. For example, at 604, the MA computing system 120 may determine comprehension tests with an average or mean score below a predetermined threshold. These tests may then be flagged and examined to determine whether the questions are difficult to understand, or whether the educational materials about the subject matter need to be improved. In another example, at 606, parameters relating to healthcare providers may be correlated to high or low scores. For example, the MA computing system 120 may determine that certain healthcare practices have consistently low scores on comprehension tests. The healthcare practices may be informed and work to improve patient communication and education. At 608, the comprehension scores may be reevaluated periodically to determine improvements.

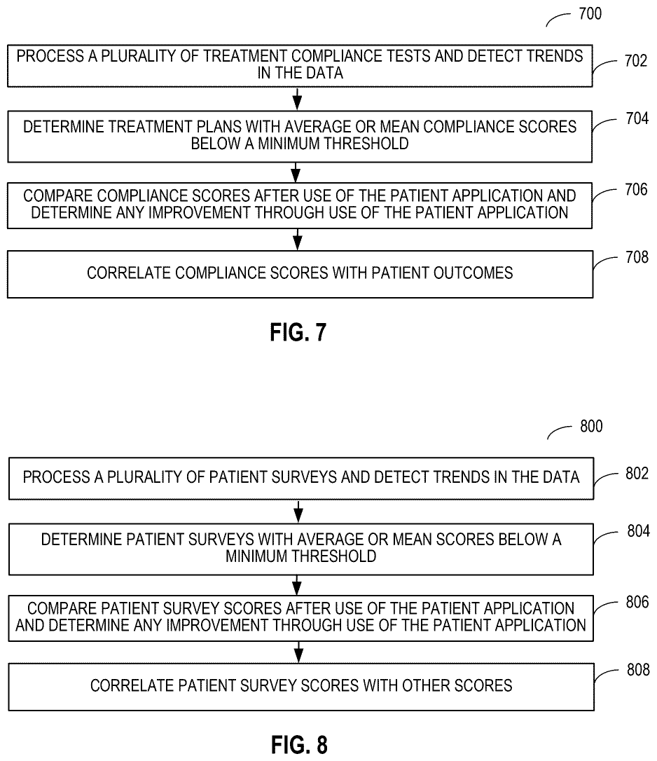

FIG. 7 illustrates a flow chart of a method 700 of the MA computing system 120 for analysing data relating to the treatment compliance questionnaires. At 702, the MA computing system 120 collates data from a plurality of treatment compliance questionnaires completed by patients across the country and determines various trends in the data. For example, at 704, the MA computing system 120 may determine compliance questionnaires that have an average or mean score below a predetermined threshold. The associated treatment plans may then be flagged and examined to determine whether the treatment plan is too difficult to follow, whether the prescribed medication in the treatment plan has a high occurrence of side effects, or whether the educational materials about the treatment plan need to be improved. In another example, at 706, the treatment compliance scores may be evaluated prior to use of the MA computing system 120 described herein and reevaluated after a predetermined period to determine improvements through use of the system.

In yet another example, at 708, the MA computing device 120 may examine data in the EMR of the patients, e.g., stored in the EMR database 126, and determine any correlation with treatment compliance scores. For example, the MA computing device 120 may determine whether patients having high treatment compliance scores have lower negative outcomes, e.g., such as a lower rate of stroke in patients with atrial fibrillation.

FIG. 8 illustrates a flow chart of a method 800 of the MA computing system 120 for analysing data relating to the patient surveys. At 802, the MA computing system 120 collates data from a plurality of patient surveys completed by patients across the country and determines various trends in the data. At 804, the MA computing system 120 may determine whether patient surveys for a particular hospital, urgent care facility, or healthcare provider are high, average or low. The healthcare providers with high patient surveys may be commended or studied to determine their patient practices. Healthcare providers or facilities with low patient surveys may be alerted. At 806, the patient survey scores may be evaluated prior to use of the patient application described herein and reevaluated after a predetermined period to determine improvements in patient satisfaction after use of the patient application. The patient survey scores may be correlated with other scores or patient outcomes at 808. For example, the MA computing system may determine whether high patient satisfaction scores correlate with high comprehension test scores, or high treatment compliance scores. In another example, the MA computing system may determine whether high patient satisfaction scores correlate with better medical outcomes for a patient, e.g., shorter hospital stays or fewer complications from surgery.

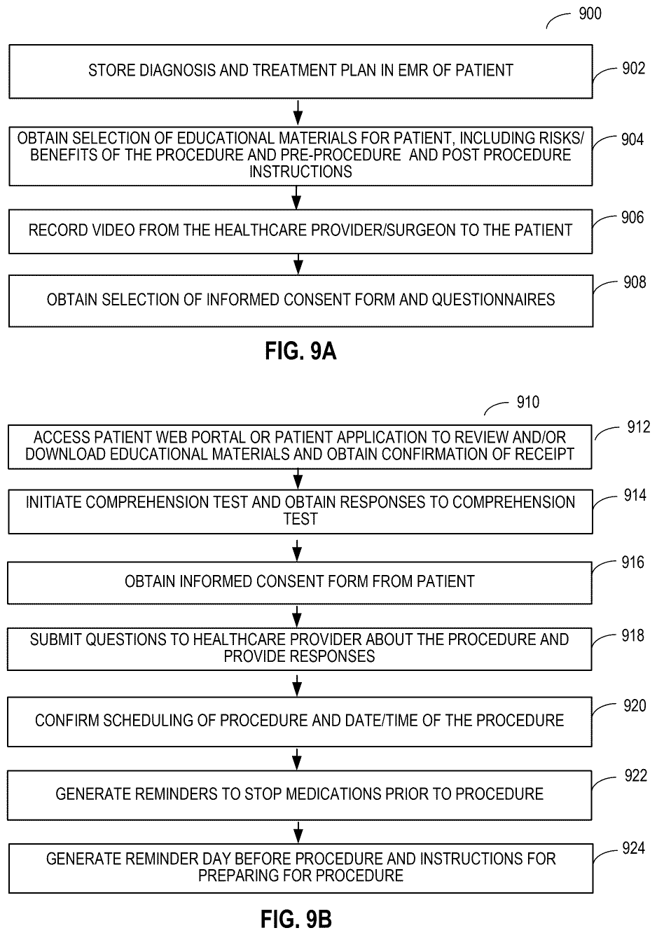

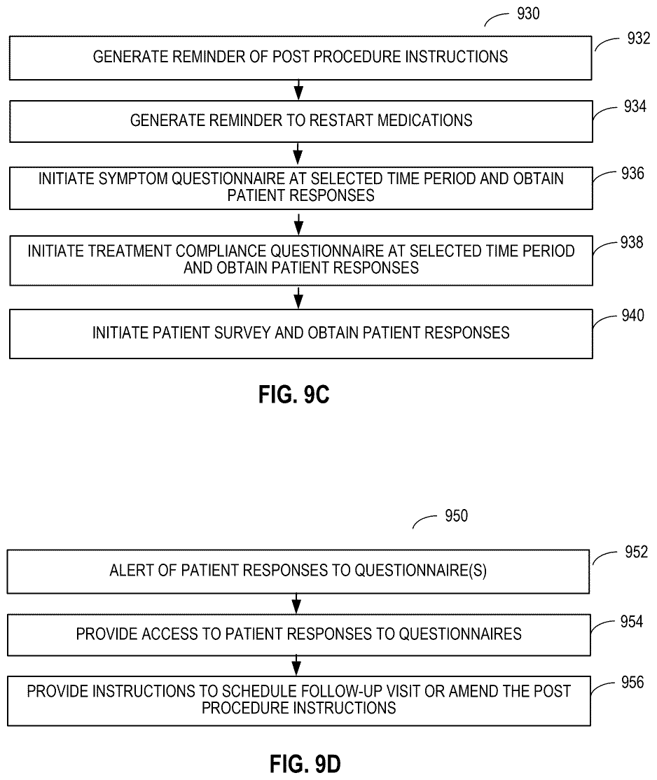

FIG. 9A illustrates a flow chart of a method 900 of the MPA computing system 120 when the treatment plan includes a patient procedure. In this example, a healthcare provider examines a patient and diagnoses cardiac conduction disease and a treatment plan including a pacemaker implant. At 902, the diagnosis and prescription are recorded in the EMR of the patient, e.g., in the EMR database 126. At 904, the healthcare provider accesses the MA computing system 120, e.g., using the web browser 112c-d or provider application 116 on a provider system 140a-c, and selects educational materials for the patient, including materials relating to the risks/benefits of the procedure, pre-procedure instructions and post procedure instructions. The risks/benefits of the procedure may include overview of the diagnosis, risks, benefits, and alternatives. The pre-procedure instructions may include instructions to stop certain medication before the procedure, such as blood thinners, diabetic medications, and blood pressure medications. In addition, the pre-procedure instructions may include requirements such as no food after midnight the night before and when and where to go for the procedure. The post procedure materials may include postoperative wound care, when to restart medications, what to expect during the healing process, symptoms to contact the healthcare provider for, follow up visits, etc.

At 906, the healthcare provider and/or the surgeon performing the procedure record a video with a personal message for the patient. The personal message inspires confidence in the diagnosis and treatment plan and helps ensure compliance with peri-procedure recommendations.

At 908, the healthcare provider selects a symptom questionnaire and time period(s) to transmit to the patient before and/or after the procedure. A treatment compliance questionnaire and time period to transmit to the patient before and/or after the procedure is also selected. The MA computing system 120 obtains the selections and provides access to the educational materials and questionnaires to the patient using the patient web portal 136 and/or patient application 114.

FIG. 9B illustrates a flow chart of another method 910 of the MPA computing system 120 prior to a patient procedure. Continuing the example from FIG. 9A, at 912, the patient accesses the educational materials relating to the diagnosis and procedure, e.g., using the patient application 114 or patient web portal 136 of the MA computing system 120. The patient may acknowledge receipt of the educational materials, e.g., using a GUI icon in the patient application 114 or web portal 136, at 912. Upon selection or request of the patient, or at a predetermined time period (such as 1-2 days) after such confirmation, the MA computing system 120 initiates a comprehension test over the provided educational materials and obtains the responses to the questions at 914.

At 916, the MA computing system 120 provides an informed consent form to the patient. The MA computing system 120 may require confirmation of receipt of the educational materials and/or completion of the comprehension test before completion of the informed consent form. The MA computing system 120 may even require a minimum score on the comprehension test prior to presenting the informed consent form. This ensures the patient understands the procedure and risks prior to agreeing to the informed consent.

At 918, the MA computing system 120 uploads questions from the patient to the provider application 116 or web portal 338. At 920, the MA computing system 120 may obtain confirmation of the scheduling of the procedure and date/time of the procedure with the patient. The schedule may be input by the healthcare provider, surgeon, and/or the patient. Using the confirmed date of the procedure and medication instructions from the healthcare provider, at 922, the MA computing system 120 may generate reminders to the patient to stop medications prior to the procedure. At 924, the MA computing system 120 may also generate reminders the day before the procedure with any instructions for preparing the procedure.

FIG. 9C illustrates a flow chart of another method 930 of the MPA computing system 120 after a patient procedure. At 932, the MA computing system 120 generates a reminder to the patient of the post procedure instructions on the procedure day and/or the days after. At 934, the MA computing system 120 also generates a reminder to restart medication on a predetermined day after the procedure. The MA computing system 120 further initiates the symptom questionnaire at the selected time period and obtains the patient responses at 936. At 938, the MA computing system 120 initiates the treatment compliance questionnaire at the selected time period and obtains the patient response. At 940, the MA computing system 120 initiates the patient survey and obtains the patient responses.

FIG. 9D illustrates a flow chart of another method 950 of the provider application or web portal of the MPA computing system 120. The MA computing system 120 may provide an alert that new patient responses are available on the provider application or web portal at 952. The healthcare provider then accesses the application and reviews the responses and/or scores of the patient questionnaires at 954. The MA computing system 120 may flag any responses that indicate a concern in the patient's condition. After review, the healthcare provider may input instructions to schedule a follow-up visit or amend the post procedure instructions.

In an embodiment, multiple healthcare providers involved in the care of the patient may input instructions, educational materials and treatment plans for a patient. The healthcare providers may thus review treatment plans from other providers to avoid any conflicting instructions. The patient has one portal to review their treatment plans and instructions rather than trying to maintain different treatment plans provided by various healthcare providers in different forms. For example, a patient is often not in a position to understand instructions from a surgeon after a procedure especially while still recovering from anaesthesia. The patient may later access the patient web portal 326 and review instructions from the surgeon and instructions from their healthcare provider. The MA computing system 120 may also alert the patient of important instructions at appropriate times, such as an alert after the procedure to restart medications.

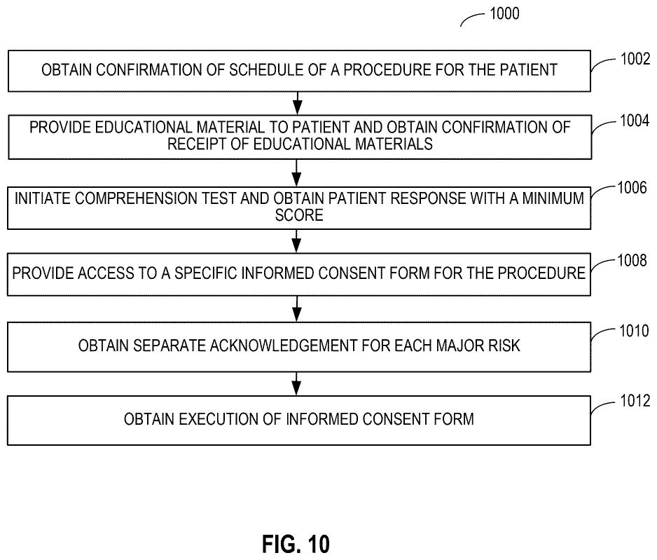

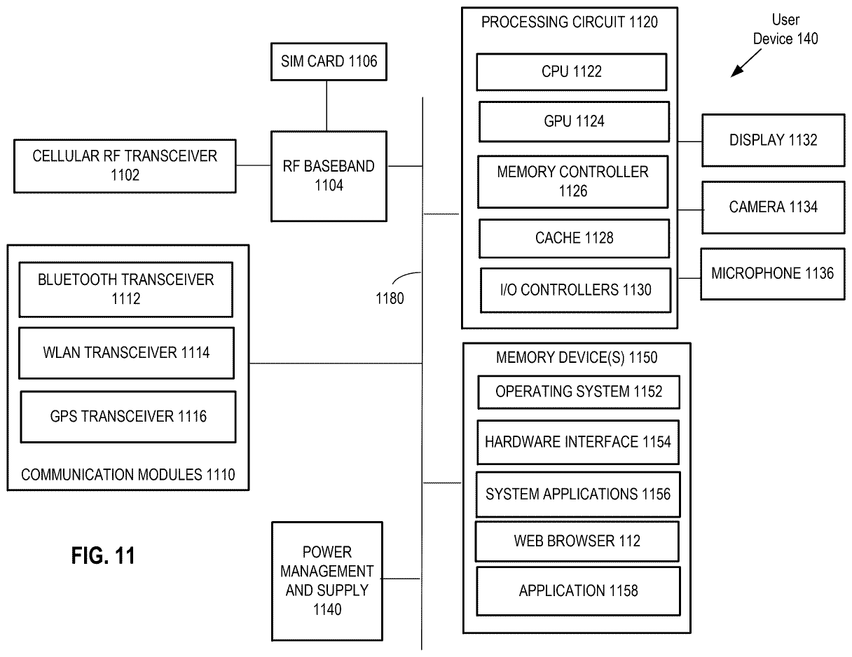

FIG. 10 illustrates a flow chart of another method 1000 of the MPA computing system 120 for obtaining informed consent of a patient. Currently, the method for obtaining an informed consent varies between providers and healthcare facilities, and the information provided to patients may not be understood or confirmed. The MA computing system 120 provides a solution for a standardized informed consent procedure. At 1002, the MA computing system 120 obtains and stores a diagnosis for a patient and a scheduled patient procedure, such as a surgery or diagnostic test. At 1004, the patient accesses the educational materials relating to the diagnosis and procedure, e.g., using the patient application 114 or patient web portal 136 of the MA computing system 120, and provides an acknowledgement of receipt of educational materials. After the acknowledgement, the MA computing system 120 initiates a comprehension test over the provided educational materials and obtains the responses to the questions at 1006. At 1008, the MA computing system 120 compares the score on the comprehension test to a minimum score. When the test score is less than the minimum score, the MA computing system 120 may generate a request for the patient to further review the materials and retake the comprehension test. In another embodiment, the MA computing system 120 may determine the missed questions and provide citation to educational materials relating to the missed questions.

When the test score is above the minimum score at 1008, the MA computing system provides access to the informed consent form. The form may be specific to the type of procedure and associated risks. At 1010, the MA computing system 120 presents the informed consent including a list of each major risk and requires acknowledgement for each major risk, such as a check mark or initial by each risk. The MA computing system 120 then obtains execution of the informed consent form at 1012.

The MA computing system 120 thus enables uniform delivery of the informed consent to patients and ensures that patients have a complete understanding of the procedure and risks. The MA computing system 120 also helps overcome barriers, such as language and age, to obtaining the informed consent.

FIG. 11 illustrates a schematic block diagram of an embodiment of a user device 1100, such as a patent device 110 or a provider system 140, used to access the MA computing system 120. In this example, the user device 1100 is a mobile device. The mobile device includes a cellular RF transceiver 1102, RF baseband 1104 and Subscriber Identity Module (SIM) card 1106 to connect to and authenticate with a cellular network for services like calls, texts, and data access. The cellular RF transceiver 1102 may be used to connect to a wide area network, such as the Internet, through the cellular network. The mobile device may include additional communication modules 1110, such as a Bluetooth transceiver 1112, WLAN transceiver 1114, and/or GPS transceiver 1116. The WLAN transceiver 1114 can also be used to connect to a wide area network, such as the Internet, e.g., through an Internet service provider.

The mobile device has one or more processing circuits 1120, such as a System-on-a-Chip (SoC), that integrate a Central Processing Unit (CPU) 1122, Graphical Processing Unit (GPU) 1124, memory controller 1126, memory cache 1128, Input/Output (I/O) controllers 1130 and/or other components. The I/O controllers 1130 interface with one or more peripherals, such as a display 1132, camera 1134, microphone 1136, etc. Power Management and Supply 1140 includes a battery and circuitry for managing power consumption and charging of the mobile device 100.

An operating system (OS) 1152, such as Android® OS developed by Google LLC or iOS® developed by Apple® Inc., is installed on the mobile device to control and operate the components of the mobile device. The operating system 1152 is stored on one or more non-volatile memory devices 1150, such as a read-only-memory (ROM). The operating system 1152, including any runtime components such as Android Runtime (ART) or iOS Runtime, executes applications on the mobile device, such as the gratitude application 118 and/or the browser 142. A hardware interface 1154 or Hardware Abstraction Layer (HAL) provides an interface between the operating system 1152 and the peripherals. System Applications 1156 includes pre-installed applications like text messenger or phone. A bus 1180 communicatively couples the various components of the mobile device.

The user device 1100 includes a browser 142 and/or application 1158, such as patient application 114 or provider application 116, to perform the functions described herein. The application 1158 may be pre-installed on the mobile device or downloaded and installed, e.g., from an application store/server such as Google Play® or Apple App Store® or from another source. In an embodiment, the application 1158 includes processor-readable instructions, data structures, program modules, application-program interfaces (“APIs”), etc. that when executed by the processing circuit 1120, causes the mobile device to perform one or more functions described herein. For example, the program modules include routines, programs, objects, components, data structures, etc. that perform tasks or implement particular abstract data types. In another embodiment, the mobile device connects to the MA computing system 120 using the web browser 112 to perform the functions described herein.

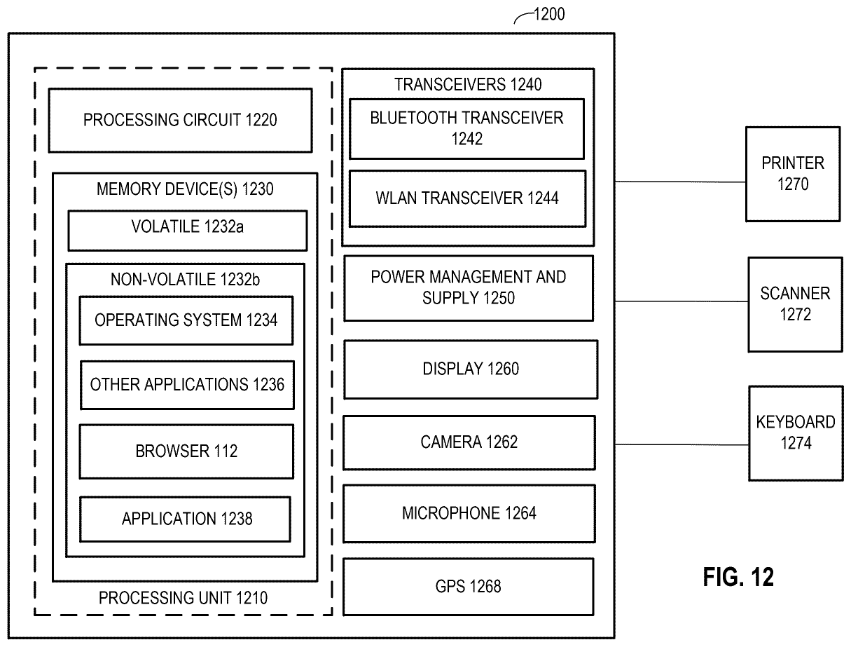

FIG. 12 depicts a schematic block diagram of an embodiment of a user device 1200, such as a patent device 110 or a provider system 140, used to access the MA computing system 120. In this example, the user device 1200 includes a personal computer or laptop. The user device 1200 includes a processing unit 1210 having at least one processing circuit 1220 and one or more memory devices(s) 1230. Depending on the exact configuration and type of the user device 1200, the memory devices 1230 may include volatile memory 1232a (such as, random access memory (“RAM”)) and non-volatile memory 1232b (such as read-only memory (“ROM”), flash memory, etc.), or some combination of the two. Additional storage may also be included, such as removable storage or non-removable storage, including, but not limited to, magnetic or optical disks or tape, thumb drives, and external hard drives, RAM, ROM, electrically erasable programmable read-only memory (“EEPROM”), flash memory or other memory technology, CD-ROM, digital versatile disks (“DVD”) or other optical storage, magnetic cassettes, magnetic tape, magnetic disk storage or other magnetic storage devices, or any other medium or any other available storage media that can be accessed by the user device 1200.

The non-volatile memory 1232b includes an operating system, such Apple®, and/or Windows® operating systems and applications 1236. One of the applications may include the browser 112 and/or application 1238, such as the patient application 114 or the provider application 116. The application 1238 includes computer-readable instructions, data structures, program modules, application-program interfaces (“APIs”), etc. that when executed by the processing circuit 1220, causes the user device 1200 to perform one or more functions described herein. For example, the program modules include routines, programs, objects, components, data structures, etc. that perform tasks or implement particular abstract data types. Such programs may be implemented in a high-level procedural or object-oriented programming language to communicate with the computer device. However, the program(s) can be implemented in assembly or machine language, if desired. In any case, the language may be a compiled or interpreted language and combined with hardware implementations. Additionally and/or alternately, the user device 1200 connects to the MA computing system 120 using the web browser 112 to perform the functions described herein.

The user device 1200 further includes one or more transceivers 1240, such as a Bluetooth transceiver 1242, WLAN transceiver 1244, and/or other wireless or wired transceivers, that allow the computing device to communicate with other devices over one or more networks. Such one or more transceivers include computer-readable instructions, data structures, program modules and/or other data, to transmit a modulated data signal such as a carrier wave or other transport mechanism and includes any information delivery media. By way of example, and not limitation, communication media includes wired media such as a wired network or direct-wired connection, and wireless media such as acoustic, radio frequency (“RF”), infrared and other wireless media. The user device 1200 may further include a power management and supply 1250 that includes a battery and components for charging and managing the batter power. The user device 1200 may further include various input/output (I/O) devices, such as a display 1260 or touch screen, touch pad, keyboard, printer, speaker, mouse, etc. The user device 1200 may also include a camera 1262 and microphone 1264 for recording patient questions or physician videos to patents, as well as a GPS system 1268. One or more peripheral devices may be coupled to the computing device, e.g., using the Bluetooth transceiver 1242, or a USB port, or other wireless or wired means, such as a printer 1270, scanner 1272 and keyboard 1274.

In the one or more embodiments described herein, the MA computing system 120 extends the contact between the patient and healthcare provider beyond the limited allotted time of a single visit. This system may be used for patient visits in a hospital, in a medical office, a telehealth appointment, urgent care visit, etc. Though healthcare visits and surgical procedure are described as examples herein, the MA computing system 120 may implement similar methods for other types of medical services, such as imaging appointments, colonoscopies, mammograms, dental appointments, etc. The system enables improved communication between the patient and the healthcare team before and after a visit, enables delivery of educational materials and other information, and fosters better patient care and satisfaction. The system also provides additional information to the healthcare provider, such as comprehension scores relating to the diagnosis and treatment plan, questionnaire responses relating to any ongoing symptoms and compliance with treatment plans, and patient survey results. This information enables more prompt follow-ups on the patient recovery and so reduces escalation of illnesses and hospital visits.

These benefits, advantages and solutions to problems described herein with regard to one or more embodiments are merely exemplary and are not required or exclusive. Any benefit, advantage, solution to a problem, or any element that may cause any particular benefit, advantage, or solution to occur or to become more pronounced are not to be construed as critical, required, or essential features or components of any or all embodiments in the claims.

Although several processes have been disclosed herein as software, it may be appreciated by one of skill in the art that the same processes, functions, etc. may be performed via hardware or a combination of hardware and software. Similarly, although the present embodiments may have been depicted as a hardwired system, these concepts may be applied to wireless systems and hybrid hardwired and wireless systems without departing from the scope of the present invention.

Some embodiments are described herein as a process that is depicted as a schematic, a flowchart, a flow diagram, a structure diagram, or a block diagram. Although a flowchart may describe the operations as a sequential process, the operations may be performed in parallel or concurrently. In addition, the order of the steps or operations may be re-arranged, and steps or operations may be combined or additional and/or alternate steps included. The described methods or process may correspond to a method, a function, a procedure, a subroutine, a subprogram, etc.

Reference to an element in the singular is not intended to mean “one and only one” unless specifically so stated, but rather “one or more.” As used herein, the terms “comprise,” “comprises,” “comprising,” “having,” “including,” “includes” or any variation thereof, are intended to reference a nonexclusive inclusion, such that a process, method, article, composition or apparatus that comprises a list of elements does not include only those elements recited, but may also include other elements not expressly listed or inherent to such process, method, article, composition, or apparatus.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A computing system including a medical application, comprising:
at least one network interface configured for communication over one or more networks;
at least one memory device, wherein the at least one memory device includes an education material database that stores a plurality of educational materials; and
at least one processing device, wherein the at least one processing device is operatively coupled to the at least one memory device and wherein the at least one memory device stores instructions that, when executed by the at least one processing device, configures the computing system to:
obtain, from a healthcare provider device associated with a healthcare provider, a selection of one or more of the plurality of educational materials for a patient, wherein one or more selected educational materials relate to a patient diagnosis or a treatment plan;
provide access to the one or more selected educational materials to a patient device associated with the patient;
generate a comprehension test relating to the patient diagnosis or the treatment plan and obtain a response to the comprehension test from the patient device;
generate a symptom questionnaire relating to the patient diagnosis or the treatment plan and obtain a response to the symptom questionnaire from the patient device;
generate a compliance questionnaire relating to patient compliance with the treatment plan and obtain a response to the compliance questionnaire from the patient device; and
provide access to the response to the comprehension test, the response to the symptom questionnaire and the response to the compliance questionnaire to the healthcare provider device.

2. The computing system of claim 1, wherein the computing system is further configured to:
obtain an upload of a video recorded by the healthcare provider to the patient; and
provide access to the video to the patient device associated with the patient.

3. The computing system of claim 1, wherein the computing system is further configured to:
obtain, from the healthcare provider device, a symptom questionnaire selection relating to the patient diagnosis and a compliance questionnaire selection relating to the patient compliance with the treatment plan.

4. The computing system of claim 1, wherein the computing system is further configured to:
obtain, from the patient device, a question for the healthcare provider;
provide access to the question to the provider device associated with the healthcare provider; and
generate an alert to the provider device.

5. The computing system of claim 1, wherein the computing system is further configured to:
generate a patient survey relating to a patient satisfaction and obtain the response to the compliance questionnaire from the patient device.

6. The computing system of claim 1, wherein the computing system is further configured to:
collate the response to the comprehension test from the patient device with a plurality of other responses to a plurality of other comprehension tests;
determine a first comprehension test of the plurality of other comprehension tests with an average score below a minimum threshold;
determine at least a first one of the plurality of educational materials relating to the first comprehension test; and
modify the at least one of the plurality of educational materials relating to the first comprehension test.

7. The computing system of claim 1, wherein the computing system is further configured to:
collate the response to the compliance questionnaire with a plurality of other responses to a plurality of other compliance questionnaires; and
determine a change in responses after a predetermined time period.

8. The computing system of claim 1, wherein the computing system further comprises:
a web server including the at least one network interface, wherein the web server comprises:
a patient web portal accessed by a first web browser on the patient device; and
a provider web portal accessed by a second web browser on the healthcare provider device; and
an application server coupled to the web server.

9. The computing system of claim 1, wherein the computing system further comprises:
an application server including the at least one network interface, wherein the patient device includes a patient application that accesses the application server using an application program interface; and wherein the provider device includes a provider application that accesses the application server using another application program interface.

10. A computing system including a medical application, comprising:
at least one network interface configured for communication over one or more networks;
at least one memory device, wherein the at least one memory device includes an education material database that stores a plurality of educational materials; and
at least one processing device, wherein the at least one processing device is operatively coupled to the at least one memory device and wherein the at least one memory device stores instructions that, when executed by the at least one processing device, configures the computing system to:
obtain, from a healthcare provider device associated with a healthcare provider, a confirmation of a scheduled procedure for a patient and a procedure date;
obtain, from the healthcare provider device, a selection of one or more of the plurality of educational materials for the patient, wherein one or more selected educational materials include risk and benefits of the scheduled procedure, pre-procedure instructions, and post-procedure instructions;
provide access to the one or more selected educational materials to a patient device associated with the patient;
generate a comprehension test relating to the one or more selected educational materials and obtain a test response to the comprehension test from the patient device; and
generate an informed consent questionnaire and obtain a questionnaire response from the patient device, wherein the informed consent questionnaire relates to the scheduled procedure.

11. The computing system of claim 10, wherein the computing system is further configured to:
obtain an upload of a video recorded by the healthcare provider to the patient; and
provide access to the video to the patient device associated with the patient.

12. The computing system of claim 10, wherein the computing system is further configured to:
obtain, from the patient device, a question for the healthcare provider;
provide access to the question to the provider device associated with the healthcare provider; and
generate an alert to the provider device.

13. The computing system of claim 10, wherein the computing system is further configured to:
determine the test response to the comprehension test from the patient device is at least a minimum score; and
generate the informed consent questionnaire for display on the patient device.

14. The computing system of claim 10, wherein the computing system is further configured to generate a reminder to the patient device of the pre-procedure instructions at least a day before the procedure date.

15. The computing system of claim 10, wherein the computing system is further configured to generate a reminder to the patient device of the post-procedure instructions on a day of the procedure date and/or a day after the procedure date.

16. The computing system of claim 10, wherein the computing system is further configured to generate a reminder to the patient device to stop one or more medications on a predetermined day before the procedure date.

17. The computing system of claim 16, wherein the computing system is further configured to generate the reminder to the patient device to restart the one or more medications on a predetermined day after the procedure date.

18. A method of a computing system, comprising:
obtaining, from a healthcare provider device associated with a healthcare provider, a selection of one or more of a plurality of educational materials for a patient, wherein the one or more selected educational materials relate to a patient diagnosis or a treatment plan;
providing, by the computing system, access to one or more selected educational materials to a patient device associated with the patient, wherein the one or more selected materials are stored in a database on a memory device of the computing system;
generating, by the computing system, a comprehension test relating to the patient diagnosis or the treatment plan for display on the patient device and obtaining a response to the comprehension test from the patient device;
generating, by the computing system, a symptom questionnaire relating to the patient diagnosis or the treatment plan for display on the patient device and obtaining a response to the symptom questionnaire from the patient device;
generating, by the computing system, a compliance questionnaire relating to patient compliance with the treatment plan for display on the patient device and obtaining a response to the compliance questionnaire from the patient device; and
providing, by the computing system, access to the response to the comprehension test, the response to the symptom questionnaire and the response to the compliance questionnaire to the healthcare provider device.

19. The method of claim 18, further comprising:
obtaining, by the computing system, an upload of a video recorded by the healthcare provider to the patient; and
providing, by the computing system, access to the video to the patient device associated with the patient.

20. The method of claim 18, further comprising:
generating, by the computing system, an informed consent questionnaire for display on the patient device and obtaining a response from the patient device, wherein the informed consent questionnaire relates to a scheduled procedure of the patient.
